(19) United States
(12) Patent Application Publication
(10) Pub. No.: US 2015/0050213 A1
(43) Pub. Date: Feb. 19, 2015

(54) COMPOSITIONS AND METHODS FOR IMAGING INFLAMMATION OF TRAUMATIC BRAIN INJURY

(71) Applicants: James R. Stone, Charlottesville, VA (US); Stuart S. Berr, Crozet, VA (US); Dongfeng Pan, Charlottesville, VA (US); Yi Zhang, Los Angeles, CA (US); Miles Lankford, Charlottesville, VA (US); Lori Nelson, North Garden, VA (US)

(72) Inventors: James R. Stone, Charlottesville, VA (US); Stuart S. Berr, Crozet, VA (US); Dongfeng Pan, Charlottesville, VA (US); Yi Zhang, Los Angeles, CA (US); Miles Lankford, Charlottesville, VA (US); Lori Nelson, North Garden, VA (US)

(73) Assignee: University of Virginia Patent Foundation, Charlottesville, VA (US)

(21) Appl. No.: 14/458,805

(22) Filed: Aug. 13, 2014

Related U.S. Application Data
(60) Provisional application No. 61/865,903, filed on Aug. 14, 2013.

(51) Int. Cl. A61K 51/08

(52) U.S. Cl. CPC A61K 51/08
USPC 424/1.69

(57) ABSTRACT

The present application provides compositions and methods for imaging traumatic brain injury using PET. The present application discloses using PET ligands targeting infiltrating neutrophils, such as a ligand of FPR, is useful for imaging inflammation. In one aspect, the ligand and imaging agent cFLFLF-PEG-⁶⁴Cu.

### CROSS REFERENCE TO RELATED APPLICATIONS

This application is entitled to priority pursuant to 35 U.S.C. §119(e) to U.S. provisional patent application No. 61/865,903, filed on Aug. 14, 2013. The entire disclosure of the afore-mentioned patent application is incorporated herein by reference.

### STATEMENT REGARDING FEDERALLY SPONSORED RESEARCH OR DEVELOPMENT

This invention was made with government support under Grant No. W81XWH-09-2-0160 awarded by Department of Defense. The government has certain rights in the invention

### BACKGROUND

High-quality non-invasive nuclear and optical imaging can be a useful diagnostic and prognostic tool for detecting sites of inflammation. This should aid in designing therapies to control pathological conditions. Although the ability to detect and characterize inflammation has been so far elusive, one of the hallmarks of inflammation is migration and activation of leukocytes (Carlos et al., 1997; Whalen et al., 1997).

Moderate to severe traumatic brain injury (TBI) is a leading cause of morbidity and mortality in military operations. One of the most significant challenges in detecting TBI is the lack of tools to establish a definitive diagnosis. In recent years, effort has been devoted to the creation of imaging agents that accumulate at sites of interest and emit a signal that can be detected by positron emission tomography (PET).

Cinnamoyl-F(D)LF(D)LFK-polyethylene glycol-⁶⁴Cu (referred to herein as cFLFLF-PEG-⁶⁴Cu) is a PET ligand for the formyl peptide receptor (FPR) which binds to circulating neutrophils, allowing for visualization of active inflammation. Zhang et al. (2007) showed that cFLFLF-PEG-⁶⁴Cu is an effective probe for in vivo imaging of acute neutrophilic inflammation (Bioorg. Med. Chem. Lett. 2007 17(24):6876-6878). They demonstrated neutrophil-specific imaging agents for detection of acute inflammation that include the neutrophil-binding peptide cinnamoyl-F(D)F(D)LFK (cFLFLFK). Several peptides targeting receptors on infiltrating leukocytes (e.g. fMLF, iBoc-MLF, and fNleLFNleYK2) have been investigated as potential imaging agents for non-invasive detection of acute inflammation. Currently used clinical nuclear imaging probes, e.g. ^{99m}Tc or ¹¹¹In labeled white blood cell and ⁶⁷Ga-citrate, need either significant preparation time and blood handling, or are not specific for inflammation. Peptide probes can specifically target neutrophils in vivo, therefore avoiding the disadvantages associated with ex vivo laboratory procedures and non-specificity. While these peptides show promise for in vivo detection of inflammation in terms of early imaging and target-to-background ratio in experimental models, several problems remain. Receptor agonist peptides may cause neutropenia or demargination due to neutrophil activation, while receptor antagonist peptides may show low uptake in infectious foci due to the low receptor affinity. The neutrophil receptor antagonist cFLFLF has been reported to have high affinity for the neutrophil N-formylpeptide receptor, but this agent has poor imaging quality due to lipophilicity resulting in high liver uptake.

Zhang et al. addressed these issues by modifying cFLFLF with biocompatible polyethylene glycol (PEG, molecular weight=3.4 k). They reasoned that the increase in hydrophilicity caused by the addition of PEG should help to attenuate hepatobiliary and intestinal uptake of the peptide. The agent they used consists of three structural components: neutrophil-specific cFLFLF; biocompatible PEG; and the radioisotope ⁶⁴Cu. The binding assay of cFLFLF-PEG-⁶⁴Cu to human neutrophils yielded Kd=5.7 nM, indicating that the PEGylation did not interfere the peptide binding with neutrophil receptor.

Tissue damage caused by trauma, infection, or other stimuli triggers a complex sequence of events collectively known as the inflammatory response. This response includes the directed migration of neutrophils from circulation to the site of injury with the ultimate goal of killing pathogens, including traumatic brain injury. Although inflammation is critical to survival, exaggerated or persistent inflammation causes collateral damage that plays a key role in the progression of many major diseases. The inflammatory response evolved to protect organisms against injury and infection. However, the powerful inflammatory response has the capacity to cause damage to normal tissue, and dysregulation of the innate or acquired immune response is involved in different pathologies.

Neutrophils are among the first leukocytes to reach a site of injury, and they are abundant at focal sites of infection. The ability to detect and quantify neutrophilic accumulation could be important, not only in locating and identifying inflammatory lesions but also in facilitating the development and testing of antiinflammatory agents (see Locke et al., 2009, J Nucl. Med. 50:790-797). Locke et al. described the synthesis and validation of a highly potent ⁶⁴Cu-labeled peptide, cFLFLFK-PEG-⁶⁴Cu, that targets the formyl peptide receptor (FPR) on leukocytes. The peptide ligand is an antagonist of the FPR, designed not to elicit a chemotactic response resulting in neutropenia. Evidence for the selective binding of this synthesized ligand to neutrophils was provided. PET properties of the compound were also shown.

There is a long felt need in the art for compositions and methods useful for detecting and diagnosing brain injury, particularly traumatic brain injury. The present application satisfies these needs.

### SUMMARY OF THE INVENTION

The present application discloses for the first time neuroimaging of inflammation in experimental traumatic brain injury. The present invention employs a fundamentally different approach to PET imaging of inflammation relative to previous work and the technique is non-invasive. Without wishing to be bound by any particular theory, this technique, as presently applied, capitalizes on the hypothesized ability of this ligand to cross the blood-cerebrospinal fluid (CSF) barrier, bound to circulating neutrophils within the periphery. The majority of PET ligands currently employed for brain imaging require the native compound to pass through the blood brain barrier, after which these compounds are able to bind to targets within the brain. However, the presently described approach relies upon the hypothesized ability for neutrophils to cross into the brain through actively crossing the blood-CSF barrier, after which they will move towards sites of injury through active chemotaxis. By binding to neutrophils within the periphery, this PET compound, which otherwise would be unable to gain access to the brain, is able to move past the blood brain /blood CSF barrier and travel to sites of injury. Therefore, in one aspect, the present invention provides compositions and methods for a non-invasive method for detecting and measuring neutrophil presence in the CSF.

In one aspect, the neutrophils and the ligand bind together in the CSF. In another aspect, they bind together and then enter the CSF. In one aspect, neutrophils that have bound ligand enter the brain via the brain-CSF barrier. In one aspect, neutrophils in the brain ventricles can be detected and measured. The neutrophils comprising a bound and labeled ligand can be detected and measured as described below. In one aspect, present invention provides compositions and methods for detecting and measuring neutrophils in the cerebrospinal fluid, comprising contacting the cerebrospinal fluid with a ligand for the formyl peptide receptor, wherein the ligand comprises a neutrophil-binding peptide linked to a hydrophilic polyethyleneglycol (PEG) moiety, and a detectable label, submitting the cerebrospinal fluid to an imaging technique and detecting and measuring neutrophils in the cerebrospinal fluid. In one aspect, the cerebrospinal fluid is a sample from a subject. In one aspect, the cerebrospinal fluid is still in the subject and a composition comprising the ligand for the formyl peptide receptor, wherein the ligand comprises a neutrophil-binding peptide linked to a hydrophilic polyethyleneglycol moiety, and a detectable label, is administered to the subject. In one aspect, an imaging technique is used to detect and measure the neutrophils in the CSF of the subject.

In one aspect, the method of the invention is useful for detecting and identifying a site of injury in the brain. In one aspect, the invention encompasses using a PET ligand targeting neutrophils.

The present application discloses the results of studies exploring the utility of cFLFLF-PEG-⁶⁴Cu as a PET ligand and imaging agent for detection of traumatic brain injury (TBI). The present application further discloses the unexpected result that the blood-brain (BB) barrier does not impede the ligand of the invention. In fact, the present invention relies on the novelty of neutrophils carrying the ligand, once attached to the cell, to a site of injury and inflammation in the brain. That is, the cell acts as a delivery system for the ligand.

In one aspect, the compositions and methods of the invention provide an imaging method that is non-invasive. In one aspect, the imaging technique is PET. In one aspect, the method detects neutrophils and/or the location of neutrophils that have migrated to the site of injury or inflammation by detecting the label attached to the ligand.

The present invention provides compositions and methods for imaging traumatic brain injury using PET. The present application discloses that using PET ligands to target infiltrating neutrophils, such as a ligand comprising a peptide that binds to the formyl peptide receptor of neutrophils, is useful for imaging inflammation. In one aspect, the ligand and imaging agent is cFLFLF-PEG-⁶⁴Cu.

In one aspect, the present invention provides for the use of ⁶⁴Cu for PET for detecting, imaging, and measuring labeled neutrophils that become associated with inflammation of brain trauma. Other radionuclides can be used as well. The method can be used to detect, localize, and quantify the amount of label present and is useful for detecting and diagnosing a site of traumatic brain injury.

One of ordinary skill in the art will appreciate that a ligand of the invention can be tagged with a fluorescent dye. In one aspect, the dye is cyanine 5.5, cyanine 7, cyanine 7.5, or indocyanine green. In one aspect, the dye is Cy5 or Cy7. In one aspect, the fluorophore is coupled to the ligand via a covalent amide bond.

The PET imaging can be coupled with other techniques, such as high-resolution laser scanning tissue imaging and cFLFLF-PEG can be successfully produced with high yield and reproducibility.

cFLFLF-PEG is useful as a ligand because it can be tagged, for example, with ⁶⁴Cu for positron emission tomography (PET) imaging. In one aspect, this molecule can also be tagged with the fluorescent dyes, including but not limited to, Cy5 and Cy7 for multiple-label immunohistochemistry and for laser scanning tissue localization respectively. The present application further discloses that cFLFLF-PEG-Cy5 demonstrates co-localization with DAPI and neutrophil elastase, confirming the specificity of this tag for infiltrating neutrophils in controlled cortical impact (CCI) traumatic brain injury.

The present application further discloses that high-resolution laser scanning tissue imaging localization of cFLFLF-PEG-Cy7 can be done within and in the periphery of contusion sites in animals undergoing controlled cortical impact traumatic brain injury. The present application further discloses the effectiveness of the technique based on tissue autoradiography following administration of cFLFLF-PEG-⁶⁴Cu, which demonstrates uptake within cortex, subcortical white matter, and peripheral deep gray matter within rodents receiving tail vein injections of this compound 12 hours prior to sacrifice at 12 hours, 24 hours, and 48 hours post-injury.

In one aspect, the present invention provides compositions and methods for localizing infiltrating neutrophils within the brain parenchyma following cortical controlled injury. The application further discloses the high yield and reproducibility of the labeled ligand, enhancing the value and efficacy of its demonstrated use herein. The compositions and methods of the invention are also useful for uptake of the ligand to tissues and areas of interest, including the cortex, subcortical white matter, and peripheral deep gray matter, and for imaging.

The present invention provides compositions and methods for detecting and diagnosing traumatic brain injury, which can be coupled with a treatment strategy, based on the imaging results. Treatment strategies can include surgery or administration of therapeutic drugs, or a combination of the two. One of ordinary skill in the art will appreciate that the treatment will vary depending on the site of the injury, the severity of the injury, and such things as the age, sex, and health of the subject.

In one aspect, the neutrophils that have bound a labeled ligand of the invention are detected by PET imaging based on the label, the amount of label measured, and then the amounts are compared to a standard level or to the levels in a non-traumatized area or to the levels in a non-traumatized subject, to make the diagnosis. In one aspect, the amounts measured are use to establish a treatment regimen.

The amount of cFLFLF-PEG-⁶⁴Cu or similar labeled ligands of the invention to be administered to a subject can vary and can be determined by one of ordinary skill in the art. It can be administered by weight/amount of ligand or by the amount of radioactivity. For example, it can be administered in amounts from about 1 μCi per 20 to 30 grams of body weight (about 33.33 μCi to about 50 μCi/kg body weight) to about 5 mCi per 20 to 30 grams of body weight (about 166.65 mCi to about 250 mCi/kg body weight). In one aspect, about 166.65 μCi to about 250 μCi/kg body weight is administered. In one aspect, about 100 μCi to about 1 mCi is administered per 20 to 30 g of body weight. In one aspect, about 3.7 to about 5.5 MBq ([˜100-150 μCi]) per 20 to 30 grams of body weight (about 5 mCi to about 7.5 mCi) can be administered. In one aspect, about 1 μCi to about 10 mCi/kg body weight or about 5 μCi to about 1 mCi/kg body weight or about 10 μCi to about 100 μCi/kg body weight or about 1 mCi to about 10 mCi/ kg body weight is administered at a time. One of ordinary skill in the art will appreciate that in one aspect, a typical does might range from about 1 to about 400 μCi/kg body weight. The ligand can be administered as a unit dose. This can be prepared in varying volumes for administration, for example, about 200 μl. Numerical ranges recited herein by endpoints include all numbers and fractions subsumed within that range (e.g., 1 to 5 includes 1, 1.5, 2, 2.75, 3, 3.90, 4, and 5). In on aspect, a unit dose of radionuclide can be administered. It is also to be understood that all numbers and fractions thereof are presumed to be modified by the term “about.” The composition of the invention can also be administered more than once and if administered more than once the intervals can vary. One of ordinary skill in the art can determined when the composition should be administered.

In one aspect, the method provides for the use of an imaging agent selected from the group consisting of a radionuclide, a radiological contrast agent, a paramagnetic ion, a metal, a biological tag, a fluorescent label, a chemiluminescent label, an ultrasound contrast agent and a photoactive agent. One of ordinary skill in the art will understand that the method of detection used will depend on the particular imaging agent used.

The invention further provides compositions and methods for monitoring the progression of inflammation or injury to the brain. The invention further encompasses monitoring a subject during treatment and after treatment.

In one aspect, a kit is provided. A kit of the invention comprises ligand of the invention that has been labeled with a detectable, an applicator, and an instructional material for the use thereof. In one aspect, the kit provides a ligand and instructions for labeling the ligand.

Various aspects and embodiments of the invention are described in further detail below.

### BRIEF DESCRIPTION OF THE DRAWINGS

FIG. 1: A schematic representation of a neutrophil imaging probe-cinnamoyl-F(D)LF(D)LFK-polyethylene glycol (cFLFLF-PEG) is a small molecule that binds to the neutrophil formyl peptide receptor (FPR). The present study explores the utility of this small molecule as a radiotracer for infiltrating neutrophil localization in TBI.

FIG. 2: Four images of fluorescent micrographs (A-D). Co-localization of DAPI (A), neutrophil elastase (B), and cFLFLF-Cy5 (C), within a neutrophil that has migrated from the periphery into the penumbra of a contusion generated through CCI TBI at 12 hours post-injury. Plates A, B, and C are fused in D.

FIG. 3: Four images of micrographs of brain sections. cFLFLF-PEG-Cy7 localized by laser scanning of tissue sections using a GE Typhoon FLA9000 system. Uptake is seen within the region of contusion at 24 hours post-injury (B), (D) that is not seen in SHAM animals (A), (C).

FIG. 4: Four images of brain sections. cFLFLF-PEG-⁶⁴Cu localized by phosphor imaging of tissue sections. Uptake is seen within the region of the contusion at 12 hours (B), 24 hours (C), and 48 hours (D) post-injury. Uptake is also seen within SHAM preparations, (A), underscoring the sensitivity of the approach. An important observation within this figure is the presence of intense uptake within the cerebrospinal fluid (CSF) occupying the visualized ventricles. This confirms neutrophils are likely entering the brain through the blood-CSF barrier, as hypothesized above with the utilization of the tracer for this disease process.

FIG. 5 comprises a schematic and six graphs (A-G). Multiple ROIs were assessed in rodents undergoing cFLFLF-PEG-⁶⁴Cu injection. Animals were assessed at 12 hours, 24 hours, and 48 hours following injury, along with SHAM controls. Mean intensity values were collected within each ROI. Contralateral matched ROIs are assessed for comparison and a ratio for each ROI was determined. Multiple statistically significant differences are seen between experimental CCI and SHAM controls. 5A—schematic of cross section of brain; 5B—Mean intensity value in the Caudate Putamen; 5C; Mean intensity value in the Hemisphere; 5D—Mean intensity value in the Hippocampus; 5E—Mean intensity value in the Corpus Callosum; 5F—Mean intensity value in the Thalamus; 5G—Mean intensity value in the Cortex. The schematic (5A), demonstrates ROIs which were utilized for determining uptake of radiotracer. Image (5B) demonstrates ratio of uptake within the caudate and putamen within the hemisphere ipsilateral to injury as compared to the contralateral hemisphere. Statistically significant peak uptake is seen at 12 hours following injury. Image (5C) demonstrates ratio of uptake within the hemisphere ipsilateral to injury as compared to the contralateral hemisphere. Statistically significant peak uptake is seen at 12 hours following injury. Image (5D) demonstrates ratio of uptake within the hippocampus within the hemisphere ipsilateral to injury as compared to the contralateral hemisphere. Statistically significant peak uptake is seen at 12 hours following injury. Image (5E) demonstrates ratio of uptake within the corpus callosum within the hemisphere ipsilateral to injury as compared to the contralateral hemisphere. Statistically significant peak uptake is seen at 12 hours following injury. Image (5G) demonstrates ratio of uptake within the cortex within the hemisphere ipsilateral to injury as compared to the contralateral hemisphere. Statistically significant peak uptake is seen at 12 and 24-hour time points following injury as compared to SHAM and 48 hours following injury. No significance was seen between the 12 and 24-hour time points.

### DETAILED DESCRIPTION

Abbreviations and Acronyms

CCI—controlled cortical impact

cFLFLF-PEG—cinnamoyl-F(D)LF(D)LFK-polyethylene glycol (note the linking lysine and that it is not used in the abbreviated name)

cFLFLF-PEG-⁶⁴Cu—cinnamoyl-F(D)LF(D)LFK-polyethylene glycol-DOTA-64Cu (note the linking lysine and DOTA and that they are not used in the abbreviated name)

CSF—cerebrospinal fluid

DOTA—1,4,7,10-tetraazacyclododecane-1,4,7,10-tetraacetic acid

FPR—formyl peptide receptor

NIF—near infrared fluorophore

PET—positron emission tomography

ROI—region of interest

SPECT—single photon emission computed tomography

TBI—traumatic brain injury

Definitions

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” a compound should be understood to mean providing a compound of the invention or a prodrug of a compound of the invention to a subject in need of treatment.

As used herein, an “agonist” is a composition of matter which, when administered to a mammal such as a human, enhances or extends a biological activity attributable to the level or presence of a target compound or molecule of interest in the mammal.

The term “alterations in peptide structure” as used herein refers to changes including, but not limited to, changes in sequence, and post-translational modification.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom or the frequency with which such a symptom is experienced by a patient, or both.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

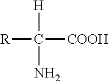

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, sputum, mucus, phlegm, tissues, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. One of skill in the art will understand the type of sample needed.

A “biomarker” or “marker” is a specific biochemical in the body which has a particular molecular feature that makes it useful for measuring the progress of disease or the effects of treatment, or for measuring a process of interest.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “compound,” as used herein, refers to any type of substance or agent that is commonly considered a drug, or a candidate for use as a drug, as well as combinations and mixtures of the above.

A “computer-readable medium” is an information storage medium that can be accessed by a computer using a commercially available or custom-made interface. Exemplary compute-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway. The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means. Compounds or agents of the invention can be administered to a subject by these means when appropriate.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

A “ligand” is a compound that specifically binds to a target receptor or target molecule.

A “receptor” or target molecule is a compound that specifically binds to a ligand.

A ligand or a receptor “specifically binds to” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels. As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

As used herein, the term “peptide ligand” (or the word “ligand” in reference to a peptide) refers to a peptide or fragment of a protein that specifically binds to a molecule, such as a protein, carbohydrate, and the like. A receptor or binding partner of the peptide ligand can be essentially any type of molecule such as polypeptide, nucleic acid, carbohydrate, lipid, or any organic derived compound. Specific examples of ligands are peptide ligands of the present inventions.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

“Pharmaceutically acceptable” means physiologically tolerable, for either human or veterinary application.

As used herein, “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder. Unless the term “treatment” or “treating” is used with the term preventive or prophylactic treatment it should not be construed to include such in less it is clear by the context.

By the term “specifically binds to”, as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds preferentially to another molecule, such as a target molecule, e.g., a nucleic acid or a protein, in the presence of other molecules in a sample..

The terms “specific binding” or “specifically binding” when used in reference to the interaction of a peptide (ligand) and a receptor (molecule) also refers to an interaction that is dependent upon the presence of a particular structure (i.e., an amino sequence of a ligand or a ligand binding domain within a protein); in other words the peptide comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

By the term “specifically binds to”, as used herein, is meant when a compound or ligand functions in a binding reaction or assay conditions which is determinative of the presence of the compound in a sample of heterogeneous compounds. The term “standard,” as used herein, refers to something used for comparison.

For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease. Unless the term “treatment” or “treating” is used with the term “preventive” or “prophylactic” it should not be construed to include such in less it is clear by the context.

### Embodiments

The imaging agent of the invention can be prepared according to the methods of Zhang et al., 2007:

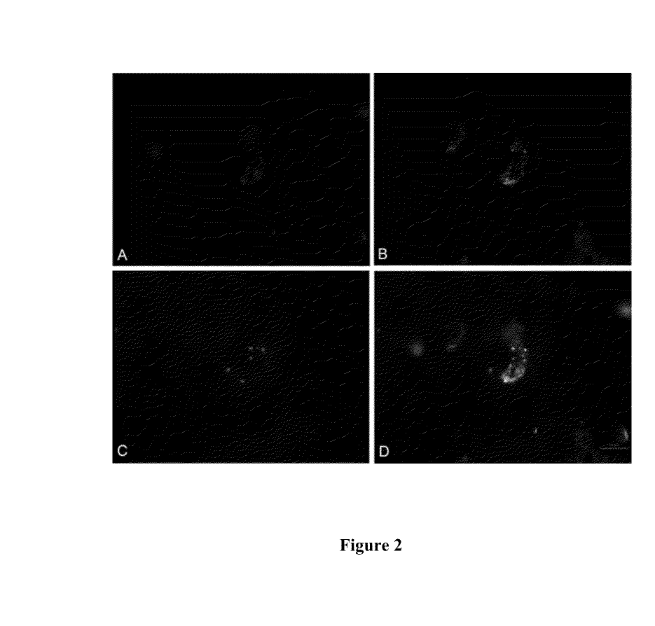

A solution of 10 mg of cFLFLFK 1, synthesized on an Advanced solid-phase peptide synthesizer by Fmoc solid phase chemistry, and 30 mg of bifunctional t-butoxycarbonyl protected PEG-succinimidyl ester (t-Boc-PEG-NHS) (MW, 3,400; Laysan Bio, Inc., USA) in acetonitrile-sodium borate buffer (0.1 N, pH 8.5) (50/50, v/v) was incubated at 4° C. overnight to yield 23 mg of cFLFLF-PEG 2 (62% from PEG), purified by HPLC and characterized by mass spectroscopy. After cleavage of the t-Boc by treating with trifluoroacetic acid (TFA) from 2, the mono-activated DOTA-SulfoNHS was coupled to the other end of the PEG moiety to produce cFLFLF-PEG-DOTA 4 which was characterized by MALDI-TOF MS (Matrix assisted laser desorption ionization time-of-flight mass spectrometry) with a number-average molecular weight of 4776. The DOTA-SulfoNHS was freshly prepared from DOTA (Macrocyclics, Inc., Dallas, Tex.), N-hydroxysulfosuccinimide (Sulfo-NHS) (PIERCE, Rockford, Ill.) and 1-ethyl-3-[3-(dimethylamino)-propyl]carbodiimide (EDC) (PIERCE, Rockford, Ill.) in aqueous (pH 5.5) at 4° C. for 30 minutes. The radiolabeling was completed by addition of 760 μCi of ⁶⁴CuCl₂ (Isotrace, Inc, O'Fallon, Mo.) to 20 μg of cFLFLF-PEG-DOTA 4 in 0.1 N ammonium acetate (pH 5.5) buffer and the mixture was incubated at 40° C. for 30 minutes. The reaction was terminated by the addition of 5 mL of EDTA solution (10 mmol/L), followed by HPLC purification (HPLC purification with a C18 reversed-phase Apollo column (5 μm, 250×10 mm). The mobile phase changed from 40% Solvent A (0.1% TFA in water) and 60% Solvent B (0.1% TFA in 80% aqueous acetonitrile) to 100% Solvent B at 30 min at a flow rate 3 mL/min. The cFLFLF-PEG-⁶⁴Cu 5 was collected with retention time at 17.2 min with radiochemical yields higher than 90%. The collected fraction was analyzed with HPLC with the same conditions (FIG. 2). The radiochemical purity was higher than 90% and the specific activity was ˜30 mCi/μmol. To characterize cFLFLF-PEG-⁶⁴Cu 5, the “cold” counterpart cFLFLF-PEG-Cu was synthesized and verified by mass spectroscopy. HPLC retention time of cFLFLF-PEG-⁶⁴Cu 5 was assigned by coinjection with the “cold” counterpart.

The following synthetic pathway is provided in Zhang et al., 2007:

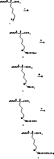

Following the synthesis of cFLFLF in 2007, Locke et al. synthesized and labeled a similar imaging agent in following manner, which is encompassed by an embodiment of the invention:

cFLFLFK-PEG-t-Boc was prepared by incubating a mixture of cFLFLFK(NH₂) (10 mg, 10.6 μmol) dissolved in 2 mL of acetonitrile and t-Boc-PEG-NHS (30 mg, 8.8 μmol) dissolved in 2 mL of sodium borate buffer (0.1N, pH 8.5) overnight at 4° C. Removal of volatiles under reduced pressure using a rotary evaporator afforded a residue that was dissolved in 2 mL of trifluoroacetic acid and left at room temperature for 2 h to remove the t-Boc protecting group. Concentration of the mixture under reduced pressure followed by reconstitution in 50% acetonitrile:water (2.0 mL) yielded stock solution. This solution was subjected to multiple injections (˜5-6) on semipreparative RP HPLC to collect fractions containing pure cFLFLFK-PEG-NH₂ (retention time, 18.4 min.). The fractions were concentrated under reduced pressure to yield pure sample, which was further characterized by MALDI-TOF mass spectroscopy. The average molecular weight distribution of cFLFLFK-PEG-NH₂ was centered at 4.3 kD, and major m/z peaks were observed at 4240, 4284, 4372, and 4416. The average calculated mass was 4331.

cFLFLFK-PEG-NH₂ (16.5 mg, 3.8 μmol) was dissolved in 1 mL of H₂O, and the pH was adjusted to 8.5 with 0.1 N NaOH. To this solution was added DOTA-NHS (19 μmol in 20 μL of water), prepared according to a previously reported method (9). The mixture was incubated overnight at 4° C. The solution was subjected to HPLC purification (retention time, 16.8 min.) to yield pure cFLFLF-PEG-DOTA (7.6 mg, 43%). Characterization of the peptide by MALDI-TOF revealed an average molecular weight distribution centered at 4.8 kD, and major m/z peaks were observed at 4644, 4688, 4776, and 4820. The average calculated mass was 4718.

The radiolabeling was accomplished by addition of 7.4-29.6 MBq (200-800 μCi) of ⁶⁴CuCl₂ to 5-20 μg of cFLFLFK-PEG-DOTA in 0.1 N ammonium acetate (pH 5.5) buffer, and the mixture was incubated at 40° C. for 30 min. The mixture was injected as is for RP HPLC purification. The column eluate was monitored by ultraviolet absorbance at 215 nm and with a y-detector. The collected product eluted at 17.2 min with a radiochemical yield higher than 95% and a specific activity of 1.1×10⁶ MBq/mmol (yield>90%). Pure fractions were concentrated under reduced pressure. The radiolabeled peptide was further characterized by comparing its chromatographic properties with nonradioactive copper-labeled compound synthesized independently using copper chloride in the same process. Analysis by MALDI-TOF revealed an average molecular weight distribution of about 4.8 kD, and major m/z peaks were observed at 4692, 4736, 4778, and 4794. The average calculated mass was 4782, which is in strong agreement with experimental values.

To test for compound stability, the compound can be incubated in serum at 37° C. for 1, 3, and 6 h. After incubation, the compound was monitored with HPLC. To determine the partition coefficient of the pegylated and nonpegylated compound, about 350 kBq of cFLFLFK-PEG-DOTA-⁶⁴Cu (or cFLFLFK-DOTA-⁶⁴Cu) was dissolved in 500 μL of water and mixed the solution with 500 μL of octanol in an Eppendorf microcentrifuge tube. The tube was sonicated for 10 min and then was centrifuged at 4,000 rpm for 5 min (Fisher Scientific Marathon Micro-A). Radioactivity was measured in 100-μL aliquots of both octanol and water layers in triplicate.

In one embodiment, cFLFLF is cF(D)LF(D)LF.

Methods for use of cFLFLF in vivo for PET imaging are also provided in Locke et al. and the amount used can be extrapolated from use in 20 to 30 gram mice to how much to use in humans:
“cFLFLFK-PEG-⁶⁴Cu (3.7-5.5 MBq [(˜100-150 μCi]) in 200 μL of saline was injected via the tail vein. Lung standardized uptake values (SUVs) were measured at several time points after injection and fit to a monoexponential curve, allowing for the calculation of ligand clearance in the control and infected lungs. This analysis provides us with an estimate of the time window after injection for which the signal difference between control and infected lungs is maximized.
For accurate image coregistration, mice were placed prone in a custom-designed portable imaging tray, facilitating precise positioning between scanners. Anesthesia (1%-2% isoflurane in oxygen) was delivered throughout the imaging. Micro-CT (14) images were acquired using a scanner developed in-house. After CT acquisition, the mice were transported to the small-animal PET scanner (Focus F-120; Siemens) and scanned for approximately 25 min. CT images were reconstructed with a 3-dimensional filtered backprojection algorithm using the COBRA software (Exxim, Inc.). The reconstructed pixel size was 0.15×0.15×0.15 mm on a 320×320×384 image matrix. Using microPET Manager (version 2.4.1.1; Siemens), PET data were reconstructed using the OSEM3D/MAP algorithm (zoom factor, 2.164). The reconstructed pixel size was 0.28×0.28×0.79 mm on a 128×128×95 image matrix. All small-animal PET images were corrected for decay but not attenuation.

Image Analysis.
PET and CT images were coregistered using ASIPRO (Siemens) and a transformation matrix previously obtained with an imaging phantom. To characterize the accumulation of the tracer in lungs, region-of-interest (ROI) analysis was performed. CT images were used to visualize lung boundaries and guide the placement of lung ROIs, which were drawn manually. Ten ±2 contiguous transaxial lung ROIs were drawn to cover the entire lung volume. Lung ROIs were transferred to the PET images, and the mean activity per milliliter of lung tissue was determined. SUVs, defined as the product of the mean lung ROI activity and the animal body weight divided by the injected dose, were computed.”

Locke et al. also showed that the clearance of cFLFLF-PEG-⁶⁴Cu in blood followed a monoexponential elimination pattern. The mean biologic half-life of the peptide was calculated to be 55±8 min. They further showed in binding studies of the peptide to human neutrophils a Kd of 17.7 nM, suggesting that the pegylation of peptide does not significantly alter the binding affinity toward the FPR, when compared with unaltered parent peptide (Kd=2 nM). Additionally, the compound cFLFLFK-PEG-⁶⁴Cu showed no biologic activity toward neutrophils as demonstrated by superoxide stimulation assays.

The present invention provides for the use of molecules such as polyethylene glycol (“PEG”) as part of the complex. One of ordinary skill in the art will appreciate that the PEG can be varied, including the use of PEGs of various molecular weights. In one aspect, the PEG is about 20,000 m.w. In one aspect, the PEG is less than 20,000 m.w. In another aspect, the PEG is less than about 18, 000 m.w. In yet another aspect, the PEG is less than about 16,000 m.w. In a further aspect, the PEG is less than about 14,000 m.w. In a further aspect, the PEG is less than about 12,000 m.w. In a further aspect, the PEG is less than about 10,000 m.w. In a further aspect, the PEG is less than about 8,000 m.w. In a further aspect, the PEG is less than about 7,000 m.w. In a further aspect, the PEG is less than about 6,000 m.w. In a further aspect, the PEG is less than about 5,000 m.w. In a further aspect, the PEG is less than about 4,000 m.w. In a further aspect, the PEG is less than about 3,000 m.w. In a further aspect, the PEG is less than about 2,000 m.w. In a further aspect, the PEG is less than about 1,000 m.w. In a further aspect, the PEG is less than about 500 m.w. In one aspect, the PEG is greater than about 500 m.w. In one aspect, PEG from about 500 m.w. to about 20,000 can be used.

Other useful techniques that can be used in the practice of the invention can be found in Zhang et al., 2007, Locke et al., 2009, and in Pan et al., U.S. Pat. Pub. No. US 2013/0144035.

An additional imaging technique can be used as well, for example, PET coupled with fluorescence. Useful techniques, depending on the label or optional label used, etc., include, but are not limited to, fluorescence, positron emission tomography (PET), magnetic resonance imaging (MRI), single photon emission computed tomography (SPECT/CT), intravital laser scanning microscopy, endoscopy, and radiographic imaging. The present technique further encompasses the use of MRI in conjunction with PET. Useful detectable labels, depending on the technique or combination of imaging techniques used, include, but are not limited to, a radionuclide, a radiological contrast agent, a paramagnetic ion, a metal, a biological tag, a fluorescent label, a near infrared label, a chemiluminescent label, an ultrasound contrast agent, and a photoactive agent.

In one aspect, using AMIDE software or a similar program, agent uptake in a region of interest such as in a region of brain traumatic injury, and remote tissue such as, liver, and muscle, is quantified with PET using co-registered, resolution-matched MR images to guide the size and location of PET ROIs. AMIDE (“A Medical Imaging Data Examiner”) is a tool for viewing, analyzing, and registering volumetric medical imaging data sets. it was developed using GTK+/GNOME, and runs on any system that supports the toolkit (Linux. Mac OS X with fink, etc.),

Optionally, a therapeutic agent can be attached or can be included in a pharmaceutical composition comprising the imaging complex.

In one aspect, the imaging agent is detected with a PET or SPECT/CT scanner coupled to a computer, and analyzing imaging data using a program.

Useful radionuclides of the invention include, but are not limited to, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²Mn, ⁵⁵Co, ⁷²As, ⁷⁵Br, ⁷⁶Br, ⁸²mRb, ⁸³Sr, or other gamma-, beta-, or positron-emitters.

In the International System of units “(SI)”, the Becquerel (Bq) is the unit of radioactivity. One Bq is 1 disintegration per second (dps). The curie (Ci) is the old standard unit for measuring radioactivity of a given radioactive sample and is equivalent to the activity of 1 gram of radium, originally defined as the amount of material that produces 3.7×10¹⁰ dps. Regarding dose levels applicable to radiopharmaceuticals, 1 GBq=27 millicuries. The rad is a unit of absorbed radiation dose in terms of the energy deposited in a living tissue, and is equal to an absorbed dose of 0.01 joules of energy per kilogram of tissue. The biologically effective dose in rems is the dose in rads multiplied by a “quality factor” which is an assessment of the effectiveness of that particular type and energy of radiation. Yet, for gamma and beta rays, the quality factor is 1, and rad and rem are equal. For alpha particles, the relative biological effectiveness (rem) may be as high as 20, so that one rad is equivalent to 20 rems. The recommended maximum doses of radiopharmaceuticals are 5 rems for a whole body dose and 15 rads per organ, while the allowable dose for children is one tenth of the adult level. The per-organ criterion protects organs where accumulation takes place. For example, radiopharmaceuticals for which removal is primarily by the liver should be administered at a lower dose than those for which removal is partly by the liver and partly by the kidney, because in the former, a single organ is involved with the removal, and in the latter, there is sharing of the removal. In order to minimize exposure to the tissue, radiopharmaceuticals, which have a long half-life, and radiopharmaceuticals, which have radioactive daughters, are generally avoided.

In one aspect, the method provides for the use of an imaging agent selected from the group consisting of a radionuclide, a radiological contrast agent, a paramagnetic ion, a metal, a biological tag, a fluorescent label, a chemiluminescent label, a near infrared label, an ultrasound contrast agent and a photoactive agent. One of ordinary skill in the art will understand that the method of detection used will depend on the particular imaging agent used.

A comparison of the levels and location in the test subject is made with the levels and location of the imaging agent from an otherwise identical location from an unaffected subject or with an unaffected area of the test subject.

In one embodiment, the imaging agent is selected from the group consisting of a radionuclide, a radiological contrast agent, a paramagnetic ion, a metal, a biological tag, a fluorescent label, a chemiluminescent label, an ultrasound contrast agent, and a photoactive agent. In one aspect, the radionuclide is selected from the group consisting of ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, and other gamma-, beta-, or positron-emitters. In one aspect, the imaging agent is detected with a PET or SPECT/CT scanner coupled to a computer, and analyzing imaging data using a program. In one aspect, the method detects the location of the inflammation in the subject. In one aspect, the invention provides for detecting and imaging a traumatic brain injury, i.e., the method can detect inflammation or trauma in multiple locations in the same subject.

Positron Emission Tomography (PET)

Positron emission tomography is a nuclear medicine imaging technique that produces a three-dimensional image or picture of functional processes in the body. The theory behind PET is simple enough. First, a molecule is tagged with a positron-emitting isotope. These positrons annihilate with nearby electrons, emitting two 511 keV photons, directed 180 degrees apart in opposite directions. These photons are then detected by the scanner that can estimate the density of positron annihilations in a specific area. When enough interactions and annihilations have occurred, the density of the original molecule may be measured in that area. Typical isotopes include ¹¹C, ¹³N, ¹⁵O, ¹⁸ F, ⁶⁴ Cu, ⁶² Cu, ¹²⁴I, ⁷⁶Br, ⁸²Rb and ⁶⁸Ga, with ¹⁸F being the most clinically utilized. Although some PET probes must be made with a cyclotron and have a half-life measured in hours, forcing the cyclotron to be on site, PET imaging does have many advantages though. First and foremost is its sensitivity: a typical PET scanner can detect between 10⁻¹¹ mol/L to 10⁻¹² mol/L concentrations.

Yet another modification may comprise the introduction of one or more detectable labels or other signal-generating groups or moieties, depending on the intended use of the labeled molecule. Suitable labels and techniques for attaching, using and detecting them will be understood by one of ordinary skill in the art, and for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be understood by the skilled artisan, and for example, include moieties that can be detected using NMR or ESR spectroscopy. Such labeled molecules of the invention may, for example, be used for in vitro, in vivo or in situ assays (including immunoassays known per se such as ELISA, RIA, EIA and other “sandwich assays,” etc.) as well as in vivo diagnostic and imaging purposes, depending on the choice of the specific label. As will be clear to the skilled person, another modification may involve the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA). Yet another modification may comprise the introduction of a functional group that is one part of a specific binding pair, such as the biotin-(strept)avidin binding pair. Such a functional group may be used to link a molecule of the invention to a protein, polypeptide or chemical compound that is bound to the other half of the binding pair, i.e., through formation of the binding pair. For example, such a conjugated molecule may be used as a reporter, for example, in a diagnostic system where a detectable signal-producing agent is conjugated to avidin or streptavidin. One non-limiting example are the liposomal formulations described by Cao and Suresh, Journal of Drug Targeting, 8, 4, 257 (2000). Such binding pairs may also be used to link a therapeutically active agent to a molecule of the invention for use in or attached to a liposome of the invention.

Imaging and Diagnostic Agents

Diagnostic agents are selected from, for example, the group consisting of a radionuclide, a radiological contrast agent, a paramagnetic ion, a metal, a fluorescent label, a chemiluminescent label, an ultrasound contrast agent and a photoactive agent. Such diagnostic agents are well known and any such known diagnostic agent may be used. Non-limiting examples of diagnostic agents may include a radionuclide such as ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, or other gamma-, beta-, or positron-emitters. Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Ultrasound contrast agents may comprise liposomes, such as gas filled liposomes. Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerythrin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

Techniques for detecting and measuring these agents are provided in the art or described herein.

Chelating Agents

In some embodiments, a chelating agent may be attached to or incorporated into a liposome of the invention, and used to chelate a therapeutic or diagnostic agent, such as a radionuclide. Exemplary chelators include but are not limited to DTPA (such as Mx-DTPA), DOTA, TETA, NETA or NOTA. Methods of conjugation and use of chelating agents to attach metals or other ligands to proteins are well known in the art (see, e.g., U.S. patent application Ser. No. 12/112,289, incorporated herein by reference in its entirety).

Various chelators can be used to make the imaging agent of the invention, including, but not limited to, DTPA, DO3A, DOTA, EDTA, TETA, EHPG, HBED, NOTA, DOTMA, DFO, TETMA, PDTA, TTHA, LICAM, HYNIC, and MECAM.

As discussed, modifications or optimizations of peptide ligands of the invention are within the scope of the application. Modified or optimized peptides are included within the definition of peptide binding ligand. Specifically, a peptide sequence identified can be modified to optimize its potency, pharmacokinetic behavior, stability and/or other biological, physical, and chemical properties.

Amino Acid Substitutions

In certain embodiments, the disclosed methods and compositions may involve preparing peptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+-0.1); alanine (−0.5); histidine (−0.5); cysteine (−1.0);

methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine;

glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

Pharmaceutical Compositions and Administration

The invention is also directed to methods of administering the compounds (ligands) of the invention to a subject. Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating a subject in need of such treatment is provided. The method comprises administering a pharmaceutical composition comprising at least one compound of the present invention to a subject in need thereof. Compounds identified by the methods of the invention can be administered with known compounds or other medications as well.

The pharmaceutical compositions useful for practicing the invention may be administered to deliver a dose of between 1 ng/kg/day and 100 mg/kg/day.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys. The invention is also contemplated for use in contraception for nuisance animals such as rodents.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

In some embodiments, the composition is administered via a route selected from the group consisting of intradermal, subcutaneous, intraperitoneal, intravenous, intraarterial, oral, and gastric routes. In some embodiments, the in vivo imaging includes but is not limited to magnetic resonance imaging (MRI), intravital laser scanning microscopy, endoscopy, PET, SPECT/CT, and radiographic imaging.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The invention also includes a kit comprising the composition of the invention and an instructional material which describes adventitially administering the composition to a cell or a tissue of a subject. In another embodiment, this kit comprises a (preferably sterile) solvent suitable for dissolving or suspending the composition of the invention prior to administering the compound to the subject.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the invention may, for example, be affixed to a container which contains the multimeric peptide of the invention or be shipped together with a container which contains the peptide. Alternatively, the instructional material may be shipped separately from the container with the intention that the instructional material and the compound be used cooperatively by the recipient.

Other techniques known in the art may be used in the practice of the present invention. In one aspect, the method is non-invasive. One of ordinary skill in the art will appreciate that the described compositions and methods can be modified and still be compatible with the practice of the invention, including, for example, changing the imaging agent, or changing the general components.

The present application further encompasses the use of other techniques for detecting, locating, or quantifying neutrophils as well as for combinations of techniques.

An additional imaging technique can be used with PET, for example, PET coupled with fluorescence. PET can also be coupled with techniques that used tissue or blood samples. Useful techniques, depending on the label or optional label used, etc., include, but are not limited to, fluorescence, positron emission tomography (PET), magnetic resonance imaging (MRI), single photon emission computed tomography (SPECT/CT), intravital laser scanning microscopy, endoscopy, and radiographic imaging. The present technique further encompasses the use of MRI in conjunction with PET. Useful detectable labels, depending on the technique or combination of imaging techniques used, include, but are not limited to, a radionuclide, a radiological contrast agent, a paramagnetic ion, a metal, a biological tag, a fluorescent label, a chemiluminescent label, an ultrasound contrast agent, and a photoactive agent.

Useful radionuclides of the invention include, but are not limited to, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, or other gamma-, beta-, or positron-emitters.

The invention is now described with reference to the following Examples and Embodiments. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

### EXAMPLES

Materials—The probe used (cFLFLF-PEG-⁶⁴Cu) has been previously described (Zhang et al.; Locke et al.). Its synthesis can be found in those publications and in part in the Embodiments herein.

### Methods

cFLFLF-PEG-⁶⁴Cu:

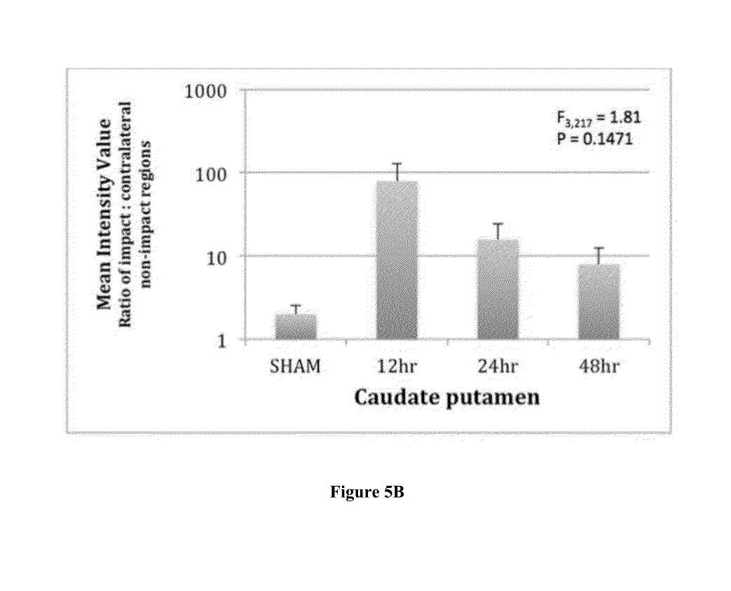

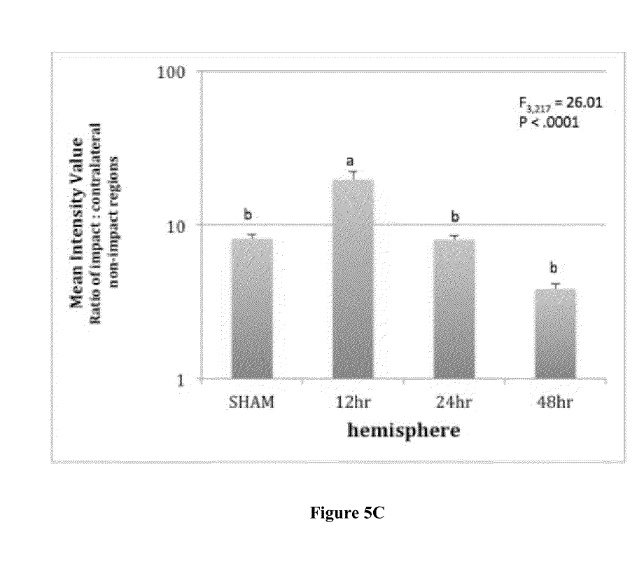

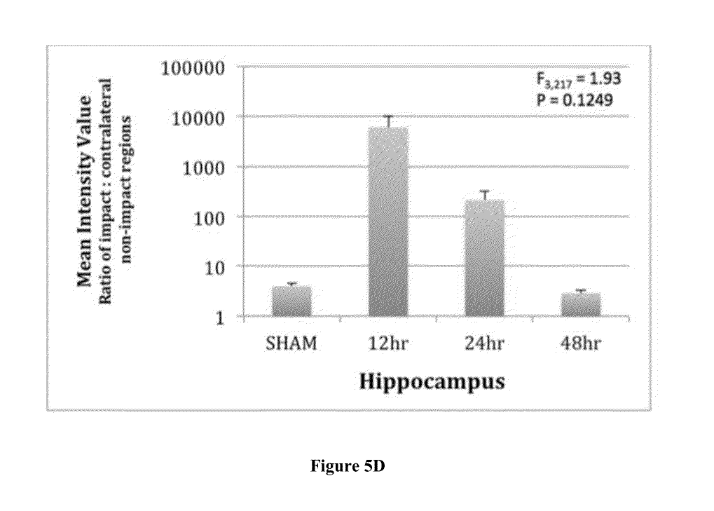

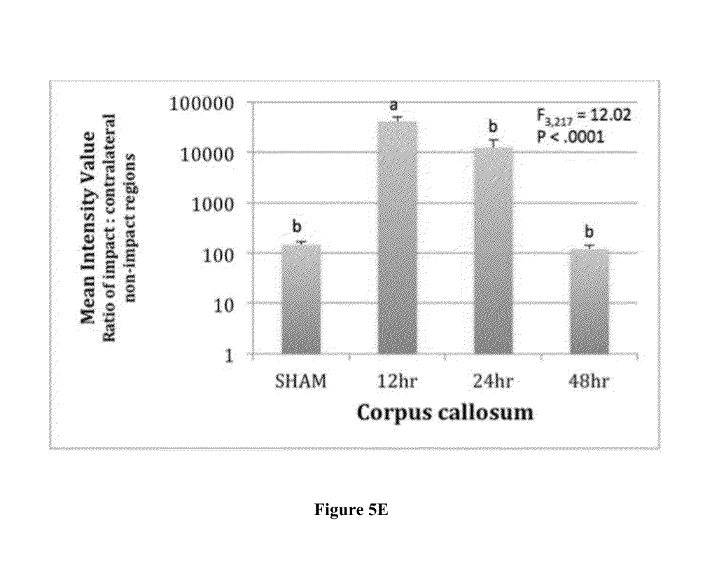

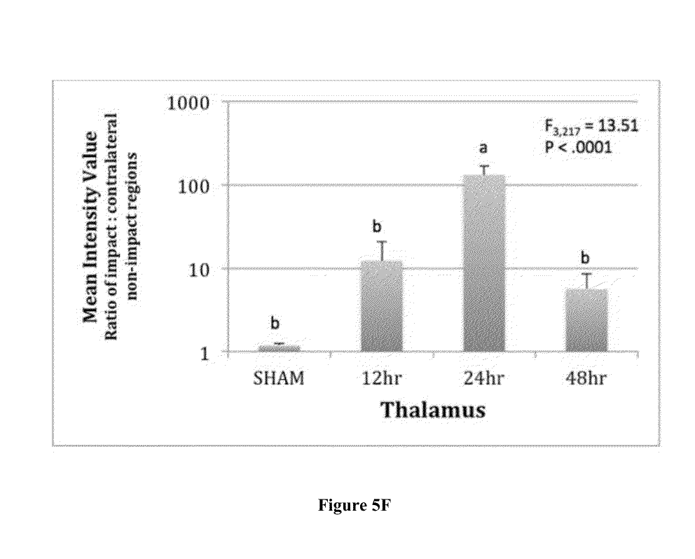

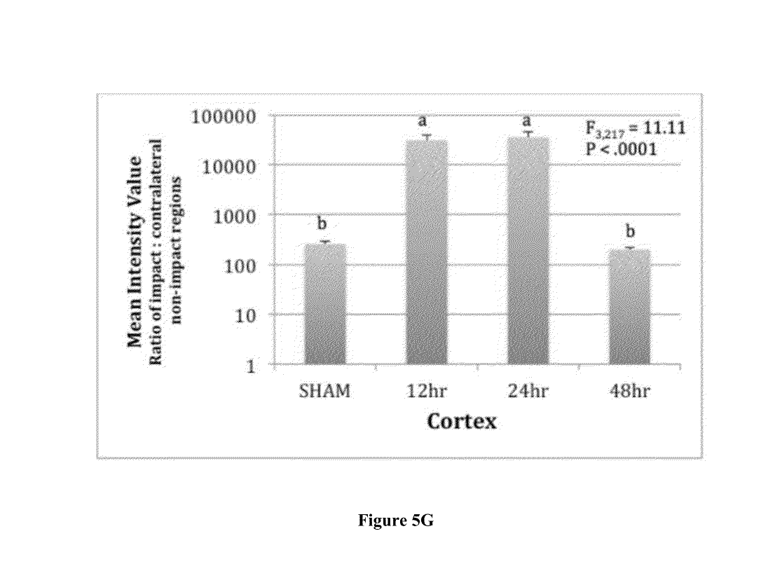

Sprague-Dawley rats (N=3/experimental group) underwent moderate-severe experimental controlled cortical impact (CCI) trauma brain injury (TBI) or SHAM preparation. Animals were sacrificed 12, 24, or 48 hours post-injury, a tail vein catheter was inserted 12 hr prior to sacrifice, and each animal received a 2 mCi dose of cFLFLF-PEG-⁶⁴Cu. Radiopharmaceutical was allowed to circulate for 12 hrs prior to sacrifice. Brains were removed and unfixed, 25 mm frozen sections were collected on a Leica CM3050S cryostat. Sections were collected onto slides and placed on radiosensitive FujiFilm SR imaging plates for 12 hours. Each screen was read with a GE Typhoon FLA9000 phosphor imager at a pixel size of 25 mm. Average intensity values from seven ROIs (FIG. 5B-5G) were measured from 20 brain sections that included the entirety of the contusion volume. Values were normalized to the corrected dose of radiation and animal body weight. Average intensity values of each ROI were compared to the contralateral anatomical region in each of the SHAM, 12, 24 hour, and 48 hour experimental groups. Phosphor imaging is a standard technique for determining whether a labeled ligand is suitable for PET imaging and positive results are an indicator that the labeled ligand is indeed useful for PET imaging.

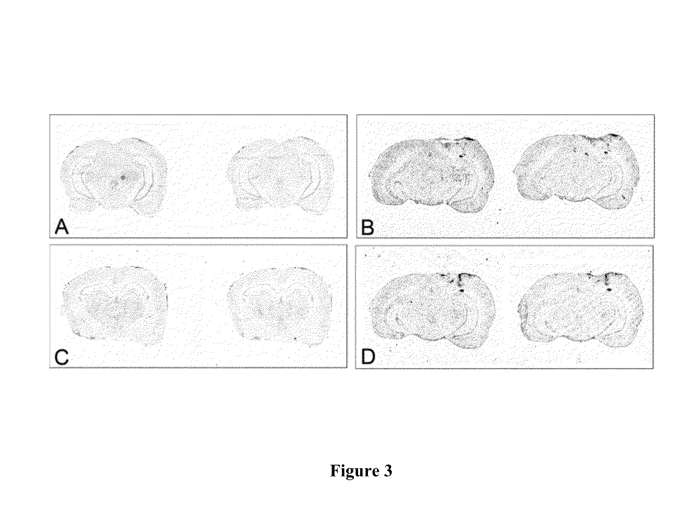

cFLFLF-PEG-Cy7: CCI injured rats and SHAM controls were injected with cFLFLF-PEG-Cy7 12 hours post-injury. Animals were sacrificed 12 hours post-injection and brain tissue was removed, sectioned as described above, and scanned with the GE Typhoon FLA9000 system using a 685 nm wavelength laser (FIG. 3).

Neutrophil co-localization: Rats underwent CCI injury as described above and were immediately injected with cFLFLF-PEG-Cy5 through a tail vein catheter. Animals were sacrificed 12 hours post-injection and perfused with 200 cc of a 10% sucrose wash and 200 cc of 4% paraformaldehyde. After four hours, animals were perfused with a 30% sucrose cryoprotectant. Brains were then removed and sectioned at a thickness of 10-18 mm. Sections were incubated in rabbit anti-rat neutrophil elastase (abcam 21595) at a dilution of 1:200 overnight at 4° C. Next, sections were washed three times in PBS and then incubated in alexa fluor 488 goat anti-rabbit antibody at a 1:200 dilution at room temperature for two hours. Sections were washed three times in PBS. Vectashield mounting medium with DAPI was applied to each section with a cover slip. Slides were assessed with a Zeiss Axioimager Z1 microscope equipped with the Apotome module. Photomicrographs were acquired using the Zeiss Zen 2011 software (FIG. 2).

### Results

1. cFLFLF-PEG can be successfully produced with high yield and reproducibility.

2. cFLFLF-PEG can be tagged with ⁶⁴Cu for positron emission tomography (PET) imaging. This molecule can also be tagged with the fluorescent dyes Cy5 and Cy7 for multiple-label immunohistochemistry and for laser scanning tissue localization respectively.

3. cFLFLF-PEG-Cy5 demonstrates co-localization with DAPI and neutrophil elastase, confirming the specificity of this tag for infiltrating neutrophils in CCI TBI.

4. High-resolution laser scanning tissue imaging demonstrates localization of cFLFLF-PEG-Cy7 within and in the periphery of contusion sites in animals undergoing CCI TBI.

5. Tissue autoradiography following administration of cFLFLF-PEG-⁶⁴Cu demonstrates uptake within cortex, subcortical white matter, and peripheral deep gray matter within rodents receiving tail vein injections of this compound 12 hours prior to sacrifice at 12 hours, 24 hours, and 48 hours post-injury. Most prominent uptake is seen at the 12 hour post-injury time point.

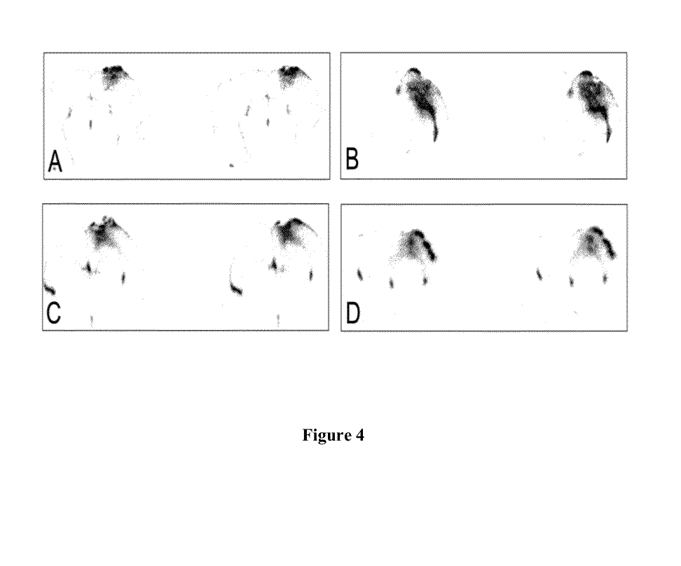

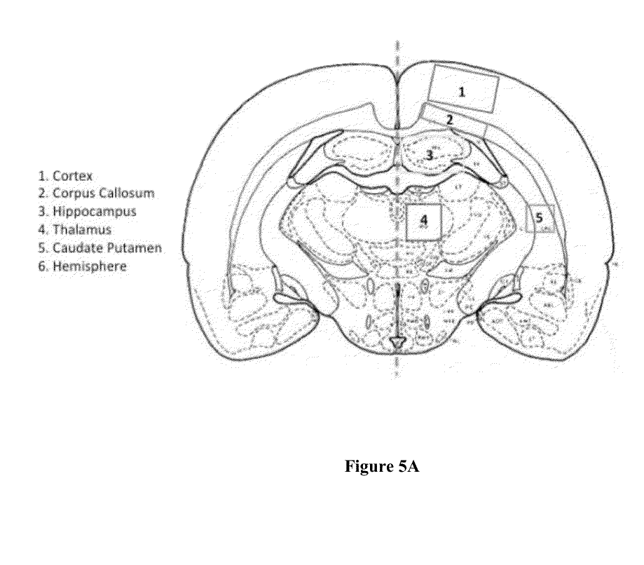

As demonstrated in FIG. 4, phosphor imaging of sections demonstrates the localization of the ligand of the invention and its usefulness. The use of phosphor imaging of tissue sections is a model useful for determining whether a probe is also useful for PET imaging.

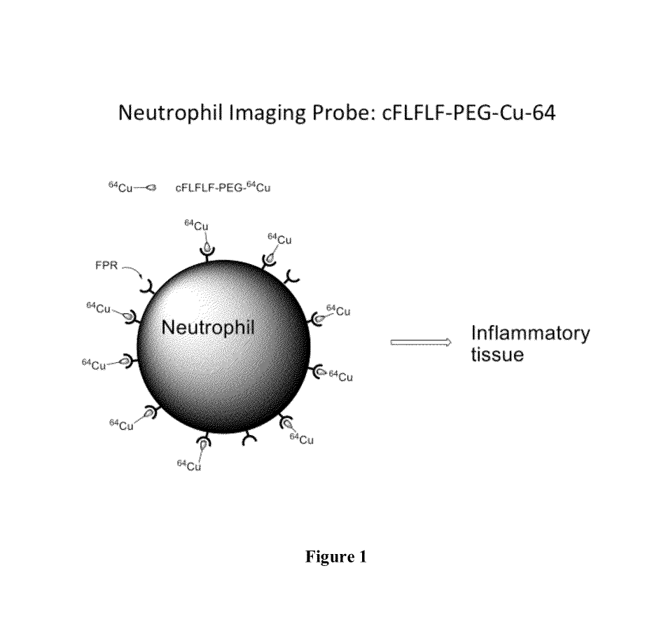

See FIGS. 1-5.

### Conclusions

cFLFLF-PEG can be produced with high yield and reproducibility. This compound localizes to infiltrating neutrophils within brain parenchyma following CCI. Uptake of cFLFLF-PEG is seen within cortex, subcortical white matter, and peripheral deep gray matter ipsilateral to injury. Future studies will explore the utility of this probe as a clinical imaging agent.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

Bibliography

Pan et al., U.S. Pat. Pub. No. US 2013/0144035 (published Jun. 6, 2013).

Zhang et al., 2007, Bioorg. Med. Chem. Lett., 17:24:6876

Locke et al., 2009, A novel neutrophil-specific PET imaging agent: cFLFLFK-PEG-⁶⁴CU, J Nucl. Med. 50:790-797.

Carlos et al., 1997, Expression of endothelial adhesion molecules and recruitment of neutrophils after traumatic brain injury in rats, Journal of Leukocyte Biology 61: 279-285.

Whalen et al., 1997, The effect of brain temperature on acute inflammation after traumatic brain injury in rats. Journal of Neurotrauma , 14: 561-572.

What is claimed is:

1. A method for imaging inflammation in the brain, said method comprising administering to a subject a composition comprising a ligand for the formyl peptide receptor, wherein said ligand comprises a neutrophil-binding peptide linked to a hydrophilic polyethyleneglycol (PEG) moiety, and a detectable label, and submitting said subject to an imaging technique.

2. The method of claim 1, wherein said detectable label is a radionuclide selected from the group consisting of ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, or other gamma-, beta-, or positron-emitters.

3. The method of claim 2, wherein said label is ⁶⁴Cu.

4. The method of claim 1, wherein the neutrophil-binding peptide is cinnamoyl-F(D)LF(D)LF.

5. The method of claim 4, wherein the neutrophil-binding peptide is linked to said PEG via a lysine.

6. The method of claim 5, wherein the neutrophil-binding peptide linked to PEG via a lysine has the formula cF(D)LF(D)LFK-PEG.

7. The method of claim 1, wherein said imaging technique is selected from the group consisting of fluorescence, positron emission tomography (PET), magnetic resonance imaging, single photon emission computed tomography (SPECT/CT), intravital laser scanning microscopy, endoscopy, and radiographic imaging.

8. The method of claim 1, wherein said inflammation is associated with traumatic brain injury

9. The method of claim 6, wherein said ligand is cFLFLF-PEG-⁶⁴Cu.

10. The method of claim 9, wherein said method detects traumatic brain injury.

11. The method of claim 1, wherein said chelating agent is selected from the group consisting of DTPA, DO3A, DOTA, EDTA, TETA, EHPG, HBED, NOTA, DOTMA, TETMA, PDTA, TTHA, LICAM, HYNIC, and MECAM.

12. The method of claim 2, wherein the radionuclide is conjugated with the ligand via a radiometal chelator or is directly coupled with a covalent bond.

13. The method of claim 9, wherein said imaging agent is detected with a PET scanner coupled to a computer, imaging data are collected, and said imaging data are analyzed using a software program.

14. The method of claim 2, wherein the radionuclide is conjugated with the imaging reagent via a radiometal chelator or is directly coupled with a covalent bond.

15. The method of claim 9, wherein uptake of said ligand is detected using PET and quantified using A Medical imaging Data Examiner (AMIDE) software using co-registered, resolution-matched magnetic resonance images to guide the size and location of PET regions of interest.

16. The method of claim 1, wherein said method is non-invasive.

17. The method of claim 1, wherein said ligand binds to a neutrophil.

18. The method of claim 17, wherein said method detects neutrophils comprising bound ligand.

19. The method of claim 17, wherein after said ligand binds to said neutrophil, the neutrophil migrates to the site of injury.

20. The method of claim 2, wherein about 1 μCi to about 10 mCi/kg body weight of radionuclide is administered.

21. The method of claim 1, wherein said method is useful for monitoring the progression or treatment of a traumatic brain injury.

22. A method of detecting and measuring neutrophils in the cerebrospinal fluid of a subject, said method comprising contacting said cerebrospinal fluid with a composition comprising an effective amount of a ligand for the formyl peptide receptor, wherein said ligand comprises a neutrophil-binding peptide linked to a hydrophilic polyethyleneglycol (PEG) moiety, and a detectable label, further wherein said ligand binds to a neutrophil, submitting said subject to an imaging technique and detecting and measuring neutrophils in the cerebrospinal fluid of the subject.
